(19) United States
(12) Patent Application Publication
(10) Pub. No.: US 2024/0293310 A1
(43) Pub. Date: Sep. 5, 2024

(54) Metal peroxide composite injectable hydrogel, preparation method and use thereof

(71) Applicant: SHENZHEN INSTITUTES OF ADVANCED TECHNOLOGY CHINESE ACADEMY OF SCIENCES, Shenzhen, Guangdong (CN)

(72) Inventors: Yuxiao LAI, Shenzhen, Guangdong (CN); Wenjing ZHANG, Shenzhen, Guangdong (CN); Wei ZHANG, Shenzhen, Guangdong (CN); Cairong LI, Shenzhen, Guangdong (CN); Zhenyu YAO, Shenzhen, Guangdong (CN)

(21) Appl. No.: 18/044,683

(22) PCT Filed: Dec. 15, 2021
(86) PCT No.: PCT/CN2021/138483

§ 371 Date: Mar. 9, 2023

(30) Foreign Application Priority Data
Nov. 15, 2021 (CN) 202111348180.9

(51) Int. Cl. A61K 9/06; A61K 9/00; A61K 38/44; A61K 47/02; A61P 19/08

(52) U.S. Cl. CPC A61K 9/06; A61K 9/0019; A61K 38/44; A61K 47/02; A61P 19/08

(57) ABSTRACT

Disclosed is a metal peroxide composite injectable hydrogel, a preparation method and use thereof. The preparation method comprises the step of dissolving a modified high-molecular polymer, a metal peroxide and a peroxidase in a solvent, mixing evenly, and standing to prepare the hydrogel, wherein the modified high-molecular polymer is a high-molecular polymer grafted with a phenolic hydroxyl group. According to the invention, the metal peroxide induces the modified high-molecular polymer to be quickly crosslinked under an action of the peroxidase to prepare the hydrogel, and the prepared hydrogel has good photo-thermal performance, antibacterial property, tissue adhesion, osteogenesis promoting effect, shape adaptability and injectability, can be well adapted to an irregular bone defect area, and is used for repairing a postoperative defect of tumor through a synergistic effect of multiple functions, which can effectively prevent tumor recurrence and reduce a possibility of implant material infection.

### CROSS-REFERENCE TO RELATED APPLICATION

This application is a national stage filing under 35 U.S.C. § 371 of international application number PCT/CN2021/138483, filed Dec. 15, 2021, which claims priority to Chinese patent application No. 202111348180.9, filed on Nov. 15, 2021. The contents of the international application are incorporated herein by reference in its entirety.

### TECHNICAL FIELD

The present invention relates to the field of biological material technologies, and particularly to a metal peroxide composite injectable hydrogel, a preparation method and usen thereof.

### BACKGROUND

Malignant tumors pose complex and diverse threats to human health. Surgical resection is widely used in current cancer treatment, but has a high risk of recurrence and may bring an inevitable damage to surrounding healthy tissues. Non-surgical treatments, such as radiotherapy and chemotherapy, also have great disadvantages, such as inflammation caused by radiation, a damage to major organs caused by drugs and a high recurrence rate^{[1]}. Bone-related malignant tumors are often accompanied by osteolytic destruction and pathological fracture, thus complicating tumor treatment. In clinical practice, doctors use large doses of antibiotics to control infection, and use artificial substitutes to repair tissue defects at the same time. This may lead to drug-resistant bacteria and inflammation in tissues around implants. Therefore, new biomedical materials and non-surgical strategies are urgently needed to prevent tumor recurrence and repair-related bone destruction. Photo-thermal therapy (PTT) is a non-invasive thermal ablation technology, which has been widely used in tumor treatment due to minimal invasion and an excellent light-induced tumor removal capability. Slight local heat (41° C. to 43° C.) may promote cell proliferation, angiogenesis, wound healing and bone regeneration. Moderate high temperature (45° C. to 50° C.) may cause a negligible damage to normal tissues and cells in a short time, but may cause a fatal damage to tumor cells. For the healing of infected wounds, thermal therapy (>50° C.) can effectively inhibit bacterial proliferation. Therefore, a photo-thermal effect may be controlled according to different temperatures for different applications^{[2]}. The photo-thermal therapy is a promising strategy to deal with multi-drug resistant bacterial infection and promote tissue regeneration.

Among many materials capable of being used for the photo-thermal therapy, an injectable polymer hydrogel has a soft property similar to an extracellular matrix (ECM), adjustable physical and chemical properties, and a capability of filling any irregular wound. The injectable viscous hydrogel can attach and bond to a defective tissue, thus accelerating the defect repair of a wound. In addition, the hydrogel may also play a role of a hemostatic agent or a sealing agent for stopping bleeding or preventing liquid or gas of the wound from leaking, and play a role of a barrier to avoid bacterial infection. Therefore, it is of great value to develop anew injectable viscous hydrogel to promote the healing of a damage of a tissue^{[2]}.

The injectable hydrogel with special functions such as photo-thermal and osteogenesis has greater transformation value and clinical significance for targeted adjuvant therapy of some diseases. In recent years, metal peroxide (MO₂) has emerged in cancer treatment. H₂O₂ can be produced by a reaction of the metal peroxide with water, wherein M is a divalent metal cation, with a reaction process of: MO₂+2H₂O→M(OH)₂+H₂O₂^{[3]}. Therefore, in an acidic micro-environment of tumor, MO₂ is easily decomposed to release metal ions and hydrogen peroxide, which may induce the overload of metal ions, the decrease of acidity and the increase of oxidative stress, with a reaction process of: MO₂+2H⁺→Mg²⁺+H₂O₂^{[4]}. In addition, the combination of MO₂ with a photosensitizer, an enzyme or a Fenton reagent may assist and promote various tumor treatments^{[5]}.

In recent years, photo-thermal nano-materials have shown a great application potential in disease treatment. The photo-thermal nano-materials have good photo-thermal conversion property and biocompatibility, convert absorbed light energy into heat energy under the irradiation of near-infrared light with a photo-thermal reagent to cause local high temperature, and kill cancer cells by utilizing a higher sensibility of the cancer cells to heat than normal cells. A nanoparticle injection strategy with the photo-thermal effect can effectively kill tumor cells to prevent tumor recurrence, but cannot undertake the function of bone defect repair caused by tumor resection.

The preparation of a peroxide/high-molecular polymer porous composite scaffold through 3D printing may also provide a treatment method for preventing tumor recurrence and bone defect repair after bone tumor resection, which can promote the bone defect repair while killing remaining tumor cells. However, the scaffold is printed and formed before implantation, and cannot adapt to an irregular tissue defect well, resulting in a cavity or a gap between the material and the tissue, thus having a risk of bacterial infection.

### REFERENCES

[1] Liu B, Gu X, Sun Q, et al. Injectable In Situ Induced Robust Hydrogel for Photothermal Therapy and Bone Fracture Repair[J]. Advanced functional materials, 2021, 31(19):2010779.
[2] Zhang X, Tan B, Wu Y, et al. A Review on Hydrogels with Photothermal Effect in Wound Healing and Bone Tissue Engineering[J]. Polymers (Basel), 2021, 13(13).
[3] Wu D, Bai Y, Wang W, et al. Highly pure MgO2 nanoparticles as robust solid oxidant for enhanced Fenton-like degradation of organic contaminants[J]. J Hazard Mater, 2019, 374:319-328.
[4] Tang Z M, Liu Y Y, Ni D L, et al. Biodegradable Nanoprodrugs: “Delivering” ROS to Cancer Cells for Molecular Dynamic Therapy[J]. Adv Mater, 2020, 32(4):e1904011.
[5] Zhu Y, Qin J, Zhang S, et al. Solid peroxides in Fenton-like reactions at near neutral pHs: Superior performance of MgO2 on the accelerated reduction of ferric species[J]. Chemosphere, 2021, 270:128639.

### SUMMARY

### Technical Problem

In the prior art, for treatment methods for bone-related malignant tumors, most scholars believe that only extensive resection and radical resection can reach an edge required by an osteosarcoma surgery. However, a bone defect is often left after bone tumor resection, and at present, commonly used materials for bone defect reconstruction in clinic comprise an autogenous bone, an allogenic bone, an artificial bone and bone cement, but various reconstruction methods have advantages and disadvantages. The bone cement is easy to use, has abundant sources, and may provide good initial stability after filling, and meanwhile, a toxic monomer and a thermal necrosis effect of an acrylic acid may also kill remaining tumor parietal cells. Therefore, methyl methacrylate bone cement is also used as a bone filling material in clinic. However, ingredients of the bone cement cannot be absorbed and reconstructed, and the bone cement has no osteogenic effect.

### Technical Solution

For the above technical problems, the present invention provides a metal peroxide composite injectable hydrogel having photo-thermal effect, a preparation method and use thereof. The hydrogel has good photo-thermal performance, antibacterial property, tissue adhesion, osteogenesis promoting effect and shape adaptability, and is used for repairing a postoperative defect of bone tumor through a synergistic effect of multiple functions, which can effectively prevent recurrence and reduce a possibility of implant material infection.

In order to achieve the above objective, the technical solution used in the present invention is as follows.

In a first aspect of the present invention, a preparation method of a metal peroxide composite injectable hydrogel is provided, which comprises the following step of:

dissolving a modified high-molecular polymer, a metal peroxide and a peroxidase in a solvent, mixing evenly, and standing to prepare the hydrogel;

wherein, the modified high-molecular polymer is a high-molecular polymer grafted with a phenolic hydroxyl group.

As a preferred embodiment, the high-molecular polymer is any one or more selected from the group consisting of gelatin, hyaluronic acid, collagen, silk fibroin, chitosan, sodium alginate, polymethacrylic acid and poly (acrylamine hydrochloride).

As a preferred embodiment, a donor of the phenolic hydroxyl group is any one or more selected from the group consisting of dopamine, 3-(4-hydroxyphenyl)propionic acid and tyramine.

As a preferred embodiment, the metal peroxide is any one or more selected from the group consisting of magnesium peroxide, calcium peroxide and zinc peroxide, and is preferably magnesium peroxide.

As a preferred embodiment, the peroxidase is horseradish peroxidase.

As a preferred embodiment, a mass ratio of the modified high-molecular polymer, the metal peroxide and the peroxidase is (150-200):(5-200):0.32.

In some specific embodiments, the mass ratio of the modified high-molecular polymer, the metal peroxide and the peroxidase is 150:200:0.32, 175:200:0.32, 200:200:0.32, 150:5:0.32, 175:5:0.32, 200:5:0.32, or any ratio among them.

As a preferred embodiment, the solvent is water or a phosphate buffered saline solution (PBS).

As a preferred embodiment, the modified high-molecular polymer is dissolved in the solvent under heating, and a heating temperature is preferably 25° C. to 50° C.

In some specific embodiments, the heating temperature is 25° C., 27° C., 30° C., 32° C., 35° C., 37° C., 40° C., 42° C., 45° C., 47° C., 50° C., or any temperature among them.

As a preferred embodiment, the metal peroxide is dissolved in the solvent under ultrasound.

As a preferred embodiment, a preparation method of the modified high-molecular polymer comprises the following step of: dissolving the high-molecular polymer in water, removing oxygen, adding 1-(3-dimethylaminopropyl)-3-ethylcarbodiimide hydrochloride (EDC), N-hydroxysuccinimide (NHS) and the donor of the phenolic hydroxyl group, reacting in inert gas, dialyzing, and drying to obtain the modified high-molecular polymer.

Preferably, the reacting in the inert gas lasts for 6 hours to 12 hours.

Preferably, in the dialyzing, a molecular weight cut-off is 8,000 to 14,000.

Preferably, the drying is freeze drying.

The EDC and the NHS are mainly used as a carboxyl activator and a cross-linking agent.

In the technical solution of the present invention, the metal peroxide reacts with water to produce H₂O₂, H₂O₂ may be decomposed into oxygen and water by catalysis of the peroxidase, and this process may be realized quickly in the phosphate buffer solution. Meanwhile, in the existence of the hydrogen peroxide, an enzymatic cross-linking reaction of the peroxidase oxidizes and converts the phenolic hydroxyl group on the modified high-molecular polymer into an o-quinone group, thus realizing cross-linking and gel forming, and endowing the hydrogel with a photo-thermal effect at the same time. A polymer network in the hydrogel provided by the present invention is formed by cross-linking a C—C bond between adjacent carbons atoms or a C—O bond between o-carbon and phenol oxygen on the aromatic ring of phenolic compounds.

In a second aspect of the present invention, a metal peroxide composite injectable hydrogel obtained by the preparation method above is provided.

In a third aspect of the present invention, use of the metal peroxide composite injectable hydrogel above in preparing a biological scaffold material is provided.

Preferably, the use refers to use in preparing a bone repair scaffold material.

In the technical solution of the present invention, an amount of the metal peroxide in the metal peroxide composite injectable hydrogel is expressed by a mass of the metal peroxide/a volume of the hydrogel, and is 5 mg/L to 200 mg/L, such as 5 mg/mL, 25 mg/mL, 50 mg/mL, 100 mg/mL, 150 mg/mL, 200 mg/mL, or any amount among them.

In the technical solution of the present invention, the metal peroxide reacts with water to produce H₂O₂, and then H₂O₂ is further decomposed into oxygen by catalysis of the peroxidase, thus realizing self-supply of O₂ to alleviate an anoxic condition of a tumor micro-environment and reconstruct the tumor micro-environment.

As a preferred embodiment, the metal peroxide is magnesium peroxide.

In the technical solution of the present invention, the magnesium peroxide is selected as the metal peroxide, which has an obvious advantage of slowly releasing and delivering Mg²⁺. Mg²⁺ is an important divalent ion during formation of biological apatite, and actively participates in control of bone formation and bone absorption. In addition, Mg²⁺ can promote osteogenic differentiation of bone marrow mesenchymal stem cells (BMSCs), enhance adhesion and diffusion of osteoblasts, promote mineralization, and participate in bone development. Moreover, Mg²⁺ can also improve cell mobility and induce cell migration, which also plays an important role in stem cell recruitment, so that the magnesium peroxide can participate in bone repair to promote osteogenic differentiation and bone regeneration of stem cells.

### Beneficial Effects of Invention

The above technical solution has the following advantages or beneficial effects.

The present invention provides the injectable hydrogel formed by mediating the phenolic-hydroxyl-modified polymer with the metal peroxide, the metal peroxide capable of producing the hydrogen peroxide induces rapid cross-linking of phenolic-hydroxyl-modified high molecules, based on the modified high molecules, the injectable hydrogel with photo-thermal tumor killing, antibacterial and anti-infection effects is formed by oxidative coupling of phenolic hydroxyl groups on the modified high molecules under the catalysis of the peroxidase. The hydrogel has good injectability, shape adaptability, photo-thermal performance, antibacterial property, tissue adhesion and osteogenesis promoting effect, can be well adapted to an irregular bone defect area, and is used for repairing a postoperative defect of bone tumor through a synergistic effect of multiple functions, which can effectively prevent recurrence and reduce occurrence of implant material infection.

### BRIEF DESCRIPTION OF THE DRAWINGS

FIG. 1 shows a preparation process of a hydrogel in Example 1 and a subsequent research project thereof.

FIG. 2a shows the injectability and shape adaptability test results of the composite hydrogels with different concentrations of metal peroxides formed by inverted gel-forming in Example 1; FIG. 2b shows the injectability test results of the composite hydrogels prepared in Example 1; FIG. 2c shows a gel forming product formed by injecting pre-gelled materials into a mold; and FIG. 2d shows another gel forming product formed by injecting pre-gelled materials into another mold.

FIG. 3a is a scanning electron microscope image of magnesium oxide nanoparticles; FIG. 3b is a scanning electron microscope image of the nanoparticles comprising both the magnesium oxide nanoparticles and the magnesium peroxide nanoparticles prepared in Example 1; FIG. 3c is a scanning electron microscope image of a modified gelatin hydrogel, and FIG. 3d is a scanning electron microscope image of a composite hydrogel comprising magnesium peroxide in prepared Example 1.

FIG. 4a shows a relationship graph that a surface temperature of the hydrogel was changed with a laser irradiation time, and FIG. 4b shows an infrared image of magnesium peroxide nanoparticles irradiated by the 808 nm near-infrared laser (with the power density of 1.0 W/cm²) for 5 minutes.

FIG. 5a shows that the hydrogel has good adhesion to a bone tissue; and FIG. 5b shows the tissue adhesion test results of the composite hydrogels with different concentrations of metal peroxides in Example 1.

FIG. 6 shows the tumor cell killing effect test results of the composite hydrogels with different concentrations of metal peroxides in Example 1.

FIG. 7 shows the antibacterial effect tests of the composite hydrogels with different concentrations of metal peroxides in Example 1.

FIG. 8 shows the in-vivo osteogenic effect tests of the composite hydrogels with different concentrations of metal peroxides in Example 1.

### DETAILED DESCRIPTION

### Embodiments of the Present Invention

The following embodiments are only some, but not all of the embodiments of the present invention. Therefore, the detailed descriptions in the embodiments of the present invention provided hereinafter are not intended to limit the scope of the present invention sought to be protected, but only represent the selected embodiments of the present invention. Based on the embodiments of the present invention, all other embodiments obtained by those skilled in the art without going through any creative work should fall within the scope of protection of the present invention.

In the following embodiments, magnesium oxide (MgO) nanoparticles are purchased from Nanjing XFNANO Materials Tech Co., Ltd. (article number: 100369), gelatin is purchased from Sigma (article number: V900863-500G), dopamine is purchased from Sigma (article number: H8502-100G), horseradish peroxidase (HRP) is purchased from Aladdin (article number: P105528-500 mg), rats are SD rats (9 to 10 weeks old), and mouse osteosarcoma osteoblasts are K7M2-wt cells.

### Example 1: Preparation of Hydrogel

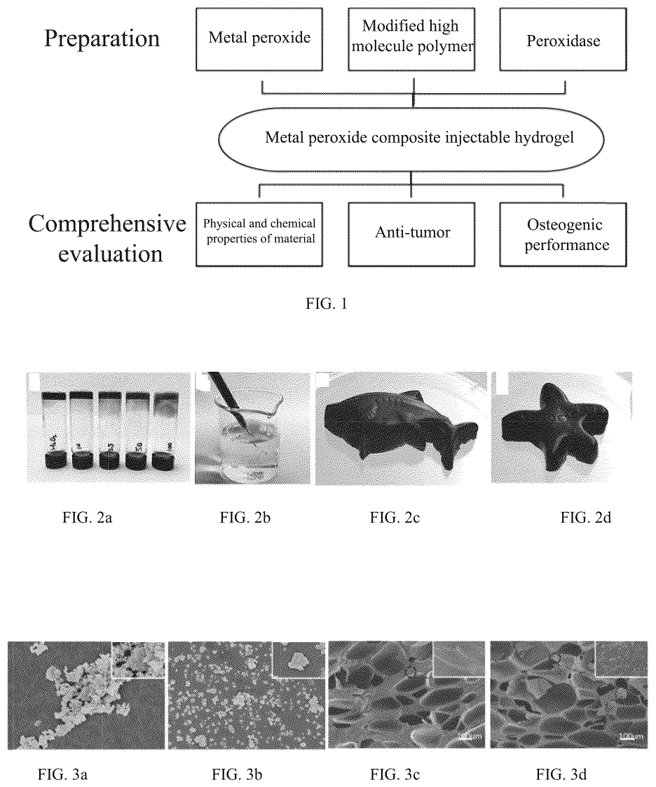

A preparation process of a hydrogel compounded with magnesium peroxide in the embodiment was shown in FIG. 1 and comprised the following steps.
1) Preparation of magnesium peroxide: 1.2 g of magnesium oxide (MgO) nanoparticles reacted with 20 ml of 30% hydrogen peroxide in anhydrous ethanol for 4 hours, with a reaction process of: MgO+H₂O₂ (30%)→2MgO₂+H₂O, and a yield of the magnesium peroxide obtained was more than 70%. Nanoparticles (comprising MgO₂ nanoparticles and some unreacted MgO nanoparticles) were obtained by drying the resultant at 70° C. for 24 hours and stored at room temperature for later use. FIG. 3a is an electron microscope image of the magnesium oxide nanoparticles, and FIG. 3b is an electron microscope image of the nanoparticles prepared in this step). As shown in FIG. 3a and FIG. 3b, under a scanning electron microscope, it could be observed that the nanoparticles prepared in this step comprised both the magnesium oxide nanoparticles and the magnesium peroxide nanoparticles.
2) Preparation of catechol-modified gelatin: 10 g of gelatin was completely dissolved in 1,000 mL of deionized water, inert gas was introduced to discharge all oxygen, then 4 g of EDC (1-(3-dimethylaminopropyl)-3-ethylcarbodiimide hydrochloride), 4 g of NHS (N-hydroxysuccinimide) and 6 g of dopamine (DA) were added, and the mixture reacted in an inert gas environment for 6 hours, and then was dialyzed by a dialysis bag with a molecular weight cut-off of 8,000 to 14,000 for 3 days, freeze-dried and then stored in refrigerator at −20° C.
3) Preparation of hydrogel: 150 mg to 200 mg of dopamine-modified gelatin (Gelda) obtained in the step 2 was completely dissolved in 0.7 mL of PBS in 40° C. water bath; the nanoparticles obtained in the step 1 were dispersed in 0.2 mL of PBS under ultrasound and mixed evenly with the modified gelatin solution above; and horseradish peroxidase was dissolved in PBS to prepare a solution with a concentration of 3.2 mg/mL, and 0.1 mL of the solution was added into the mixed solution above, mixed evenly, and stood for a few minutes to form the hydrogel. A mass ratio of raw materials of the system was that: modified gelatin:nanoparticles:HRP=(150-200):(5-200):0.32.

Results of the hydrogel prepared in the example were shown in FIG. 3d. Under a scanning electron microscope, it could be observed that, compared with the modified gelatin hydrogel in FIG. 3c (with a locally enlarged image indicated by a circled part), the magnesium peroxide nanoparticles existed in the hydrogel in FIG. 3d (as shown in a locally enlarged image indicated by a circled part).

As shown in FIG. 2 a, FIG. 2b, FIG. 2c and FIG. 2d, according to the example, an injectability and a shape adaptability of the hydrogel were proved by various gel-forming methods, wherein FIG. 2a showed inverted gel-forming, which specifically comprised evenly mixing the PBS solution added with MgO₂, HRP and the modified gelatin in the step 3, and then inverting the reaction container. In FIG. 2b, the mixed solution comprising the PBS solution of MgO₂ and the PBS solution of HRP in the step 3 was injected into the modified gelatin solution by a 1 mL syringe for gel forming, which proved the injectability of the hydrogel. In FIG. 2c and FIG. 2d, gel forming products with different shapes were formed by injecting pre-gelled materials into different molds. It could be seen from FIG. 2 a, FIG. 2b, FIG. 2c and FIG. 2d that the hydrogel provided by the present invention could realize gel forming by different methods, and had the shape adaptability.

According to a concentration representation method of the hydrogel prepared in the example: 10 MG, 25 MG, 50 MG and 100 MG respectively represents hydrogels with masses of magnesium peroxide/volumes of hydrogel of 10 mg/mL, 25 mg/mL, 50 mg/mL and 100 mg/mL.

### Control Group G:

A hydrogel in the control group G was a composite hydrogel comprising hydrogen peroxide, a preparation process thereof was the same as the above-mentioned preparation process, but the magnesium peroxide nanoparticles were replaced by the hydrogen peroxide, and the preparation process was specifically as follows:

150 mg of dopamine-modified gelatin (Gelda) obtained in the step 2 was completely dissolved in 0.7 mL of PBS in 40° C. water bath, then 0.2 mL of 0.5% H₂O₂ solution was mixed with the above solution, then 0.1 mL of horseradish peroxidase (HRP) with a concentration of 3.2 mg/mL was added, and the prepared solution was mixed evenly, and stood for several minutes to form the hydrogel.

### Example 2: Photo-Thermal Performance Test of Hydrogel

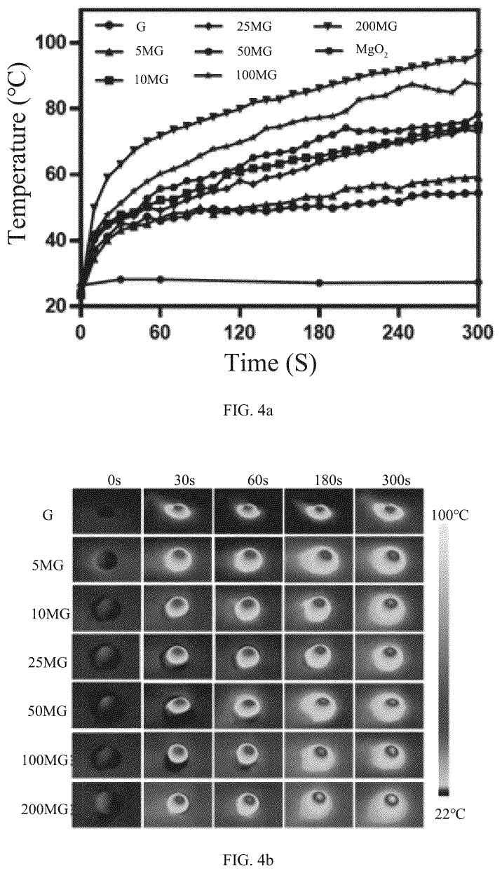

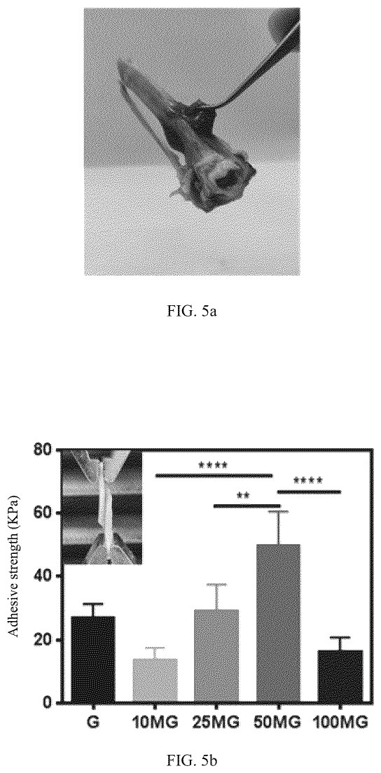

In order to verify a photo-thermal effect of the hydrogel, the hydrogel prepared in Example 1 was exposed to 808 nm near-infrared laser (with a power density of 1.0 W/cm²) for 5 minutes, and a laser spot was adjusted to completely cover a whole surface of a sample. Results were shown in FIG. 4a and FIG. 4b. FIG. 4a showed a relationship graph that a surface temperature of the hydrogel was changed with a laser irradiation time, and FIG. 4b showed an infrared image of magnesium peroxide nanoparticles irradiated by the 808 nm near-infrared laser (with the power density of 1.0 W/cm²) for 5 minutes, without producing the photo-thermal effect. FIG. 4b was the infrared image of FIG. 4a. It could be seen from FIG. 4a and FIG. 4b that the hydrogel provided by the present invention had a good photo-thermal conversion capability under the irradiation of the 808 nm laser.

### Example 3: Tissue Adhesion Test of Hydrogel

In order to test the tissue adhesion of the hydrogel, a shin bone of a hind leg of a rabbit was selected for the tissue adhesion test, 50 mg of magnesium peroxide/mL of hydrogel prepared in Example 1 was adhered to the shin bone of the hind leg of the rabbit and taken with a tweezer, and then it was found that the hydrogel was not separated from the shin bone of the hind leg of the rabbit (referring to FIG. 5a), which proved that the hydrogel had good adhesion to a bone tissue.

A fresh shaved pigskin without excess fat was obtained from the local market and cut into rectangular strips (length 25.0 mm×width 10.0 mm×thickness 2.0 mm). Before testing, the cut pigskin slices were washed with a saline solution (0.9 wt % NaCl solution) and soaked in 4.0° C. PBS buffer solution (pH=7.4) overnight to ensure that the pigskin samples remained moist. The hydrogel prepared in Example 1 was coated on regions of about 1.0 cm×1.0 cm on surfaces of two pigskin strips respectively. Then, the two pigskin strips were immediately pressed together by a constant force. Then, the adhered pigskin slices were pulled apart by a mechanical testing device at a cross-head speed of 1.0 mm/min and a gauge length of 50.0 mm, and a lap shear adhesive strength (Pa) was calculated by dividing a maximum load (n) by an overlapped area (m²) of an adhesive. Results were shown in FIG. 5b. The composite hydrogels with different concentrations of magnesium peroxide in Example 1 and the composite hydrogel comprising the hydrogen peroxide in Comparative Example 1 all had excellent tissue adhesion, wherein lap shear adhesive strengths of 10 MG and 100 MG hydrogels were weaker than that of Comparative Example 1, while lap shear adhesive strengths of 25 MG and 50 MG hydrogels were far higher than that of the control group G, and the lap shear adhesive strength of 50 MG hydrogel had a highest lap shear adhesive strength.

### Example 4: Osteogenesis Promoting Effect of Hydrogel

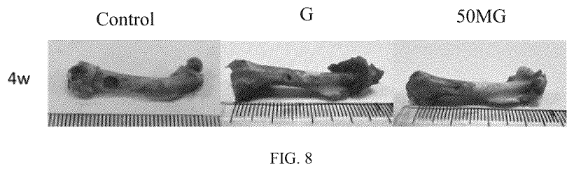

After a rat was anesthetized by intraperitoneal injection of 2.5% pentobarbital sodium (40 mg/kg body weight), a femoral defect with a diameter of 3 mm was slowly drilled by an electric drill (Kingrock Medical Instrument, China, Shanghai). In this process, a part of periosteum was removed, and a drill hole was washed with a saline solution and then cleaned by suction. A composite hydrogel comprising magnesium peroxide at a concentration of 50 mg/mL was injected into the defect, and an incision was sutured and thoroughly cleaned with a povidone iodine disinfectant. Penicillin was administrated every day for 3 days after operation. Four and eight weeks after material implantation, the repair of the bone defect of the rat was observed. Preliminary results of the repair of the bone defect 4 weeks later were shown in FIG. 8. It could be seen from the figure that, compared with a defect region of a bone defect part implanted with the composite hydrogel comprising peroxide hydrogel in the control group G, and the control group (a blank control without material implantation after hole drilling), a defect region of a bone defect part implanted with the composite hydrogel comprising magnesium peroxide (a 50 MG group) was obviously smaller, indicating that the defect was repaired to a certain extent.

### Example 5: Tumor Recurrence Resistance Property of Hydrogel

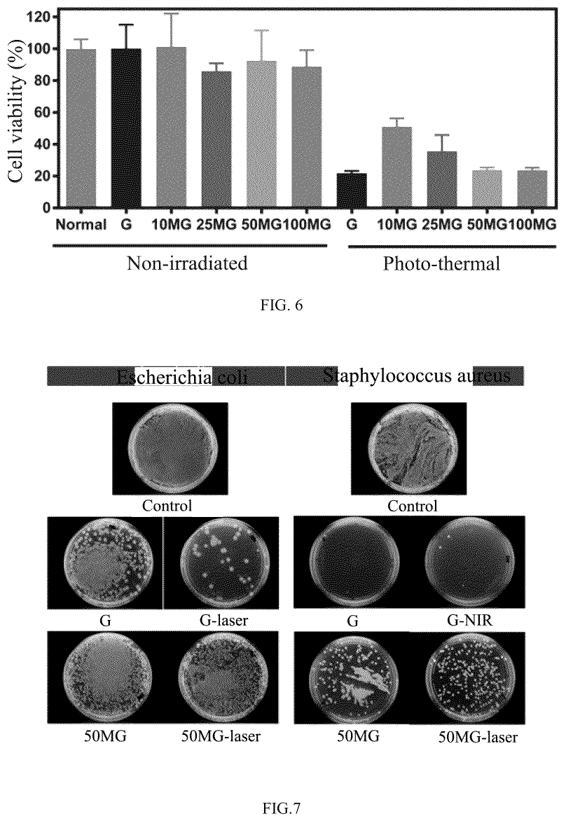

A CCK8 method was used to detect a photo-thermal cytotoxicity of the hydrogel in Example 1 to K7M2-wt cells. After 10,000 cells were inoculated in a 48-well plate and adhered overnight, a supernatant in each well was taken out, the cells were covered with the hydrogel, and a small amount of 1640 culture medium was supplemented. After the hydrogel was irradiated by 808 nm laser (1 w/cm²) for 5 minutes, the hydrogel and the supernatant were taken out, the culture medium was replaced, and the cells were incubated again for 24 hours. Then, 200 L of culture medium containing 10% CCK8 was added into each well. After incubation for 1 hour, an absorbance at 450 nm was determined by a microplate reader. Relative cell viability (%)=(average OD450 sample/average OD450 PBS)*100. Results were shown in FIG. 6 (wherein Normal referred to normal untreated cells), and an ordinate indicated a cell viability. Under a photo-thermal condition (laser+), the hydrogel in Example 1 had a very obvious tumor cell killing effect, and the killing effect was enhanced when a concentration of the hydrogel was increased; while under a non-irradiated condition (laser−), the tumor cells survived well, with a cell viability higher than 80%.

### Example 6: Antibacterial Property of Hydrogel

Under irradiation of 808 nm laser, Escherichia coli and Staphylococcus aureus were incubated on a surface of the hydrogel prepared in Example 1 for 2 hours, and then an antibacterial effect of the material was observed by a plate colony counting method. Results were shown in FIG. 7. The hydrogel group in the control group G and the hydrogel group (50 MG) in Example 1 both had certain inhibitory effects on Escherichia coli (E. coli) and Staphylococcus aureus (S. aureus) under a non-irradiated condition, and the effect on Staphylococcus aureus was better than that on Escherichia coli.

The above are only preferred embodiments of the present invention, which do not limit the patent scope of the present invention. Any equivalent transformation made according to the contents of the specification and the drawings of the present invention, or directly or indirectly applied in related technical field, is similarly included in the scope of protection of the patent of the present invention.

1. A preparation method of a metal peroxide composite injectable hydrogel, comprising:
dissolving a modified high-molecular polymer, a metal peroxide and a peroxidase in a solvent, mixing evenly, and standing to prepare the hydrogel;
wherein, the modified high-molecular polymer is a high-molecular polymer grafted with a phenolic hydroxyl group.

2. The preparation method according to claim 1, wherein the high-molecular polymer is any one or more selected from the group consisting of gelatin, dopamine, 3-(4-hydroxyphenyl)propionic acid, tyramine, hyaluronic acid, collagen, silk fibroin, chitosan, sodium alginate, polymethacrylic acid and poly (acrylamine hydrochloride).

3. The preparation method according to claim 1, wherein the metal peroxide is at least one selected from the group consisting of magnesium peroxide, calcium peroxide and zinc peroxide.

4. The preparation method according to claim 1, wherein the peroxidase is horseradish peroxidase.

5. The preparation method according to claim 1, wherein a mass ratio of the modified high-molecular polymer, the metal peroxide and the peroxidase is (150-200):(5-200):0.32.

6. The preparation method according to claim 1, wherein the solvent is water or a phosphate buffered saline solution.

7. The preparation method according to claim 1, wherein a preparation method of the modified high-molecular polymer comprises the following step of: dissolving the high-molecular polymer in water, removing oxygen, adding 1-(3-dimethylaminopropyl)-3-ethylcarbodiimide hydrochloride (EDC), N-hydroxysuccinimide (NHS) and the donor of the phenolic hydroxyl group, reacting in inert gas, dialyzing, and drying to obtain the modified high-molecular polymer.

8. A metal peroxide composite injectable hydrogel obtained by the preparation method according to claim 1.

9. A biological scaffold material, comprising the metal peroxide composite injectable hydrogel according to claim 8.

10. The biological scaffold material according to claim 9, wherein the metal peroxide is magnesium peroxide.

11. The preparation method according to claim 1, wherein a donor of the phenolic hydroxyl group is any one or more selected from the group consisting of dopamine, 3-(4-hydroxyphenyl)propionic acid and tyramine.

12. The preparation method according to claim 3, wherein the metal peroxide is magnesium peroxide.

13. The preparation method according to claim 7, wherein the reacting in the inert gas lasts for 6 hours to 12 hours.

14. The preparation method according to claim 7, wherein in the dialyzing, a molecular weight cut-off is 8,000 to 14,000.

15. The preparation method according to claim 7, the drying is freeze drying.

16. The biological scaffold material according to claim 9, wherein the biological scaffold material is a bone repair scaffold material.
